(19) United States
(12) Patent Application Publication
(10) Pub. No.: US 2022/0387129 A1
(43) Pub. Date: Dec. 8, 2022

(54) SYSTEM, METHOD AND COMPUTER PROGRAM PRODUCT FOR IMPROVED MINI-SURGERY USE CASES

(71) Applicant: PATHKEEPER SURGICAL LTD., Rehovot (IL)

(72) Inventor: Opher KINROT, Ra'anana (IL)

(21) Appl. No.: 17/776,218

(22) PCT Filed: Nov. 12, 2020
(86) PCT No.: PCT/IL2020/051173

§ 371 Date: May 11, 2022

Related U.S. Application Data
(60) Provisional application No. 62/934,021, filed on Nov. 12, 2019.

(51) Int. Cl. A61B 90/00; A61B 34/20; A61B 8/00

(52) U.S. Cl. CPC A61B 90/361; A61B 90/37; A61B 90/39; A61B 34/20; A61B 8/4254; A61B 8/4444; A61B 2090/363; A61B 2090/371; A61B 2090/3762; A61B 2090/3916; A61B 2090/3937; A61B 2034/2057; A61B 2034/2065

(57) ABSTRACT

An imaging system aka 3d camera operative in conjunction with a tube having two open ends, the system comprising active portions small enough to fit into the tube and an electronic subsystem including a hardware processor operative to receive image/s from the active portions and to generate therefrom at least one 3D image of a scene visible via one of the tube's open ends. The system may comprise a tracker configured to be secured to the tube, and a method for monitoring location, e.g. absolute location, of the tube, accordingly.

### REFERENCE TO CO-PENDING APPLICATIONS

Priority is claimed from U.S. provisional application No. 62/934,021, entitled System, method and computer product for improved minimally invasive spine surgery” and filed Dec. 11, 2019, the disclosure of which application/s is hereby incorporated by reference.

### FIELD OF THIS DISCLOSURE

The present invention relates generally to imaging systems, and more particularly to image-guided techniques.

### BACKGROUND FOR THIS DISCLOSURE

Mini-surgery is an important new field. For example, Minimally Invasive Spine Surgery (MISS) is a growing trend in spine surgery, currently stronger in simpler and common lumbar fusion surgery. A review of the evolution of MISS procedures is given in Reference 1 https://doi.org/10.3171/2018.11.SPINE181215.

MISS procedures are designed to reduce collateral damage to muscles and ligaments, as well as blood loss. These factors help reduce patient recovery time following the surgery. The Minimally Invasive Spine Surgery (MISS) procedure typically uses several smaller incisions located directly where the implant goes into the vertebrate (instead of one larger incision). For example, in a 2-level fusion where 2 vertebrae are fused together, the surgeon may need to implant:

4 screws (two in each vertebra to be fused, on both sides of the spine midline);

2 rods (each rod connects two screws on the same side of the spine midline);

1 cage (replacing the disc between the two vertebrae).

In such a surgery the surgeon will typically use one of various techniques to make a small incision, and add a retractor tube to allow for space to use surgery tools and to insert the implants. In some cases the screws can be inserted “directly” through the skin (percutaneously), however a larger incision is required for insertion and placement of the rods and cage.

In a sample MISS procedure two retractors (such as, e.g. https://www.aesculapimplantsystems.com/products/spine-solutions/thoracolumbar-solutions/spyder-mis-retractor-system) typically deployed on both sides of the spinous process, are used to approach the vertebra from both the left and right sides. The retractors are each inserted through a skin incision and their respective tubes (aka “MISS retractor tubes”) allow access to the vertebra bone on the left and right sides of the spinous process (middle bone protrusion in cross-sectional view).

‘Range imaging’, according to Wikipedia, is “a collection of techniques that are used to produce a 2D image showing the distance to points in a scene from a specific point, normally associated with some type of sensor device. The resulting image, the range image, has pixel values that correspond to the distance. If the sensor that is used to produce the range image is properly calibrated, the pixel values can be given directly in physical units, such as meters”.

Surface matching, and its use in surgery generally, is known, e.g. as described here: http://cobweb.cs.uga.edu/˜suchi/pubs/ananda-ieee-isbi-2004.pdf. Surface matching is also an OpenCV module.

The disclosures of all publications and patent documents mentioned in the specification, and of the publications and patent documents cited therein directly or indirectly, are hereby incorporated by reference, other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented as appropriate.

Certain embodiments seek to provide improved mini-surgery devices and processes.

Certain embodiments seek to provide a surgical imaging system comprising a miniature camera configured to capture a camera-captured representation of a 3D structure which is present at a far end of a tube, upon being secured to a near end of the tube, and/or a tracker which tracks its own position and orientation and, upon being attached to a surgical tool, derives the surgical tool's position and orientation from the tracker's own position and orientation, and/or a hardware processor in data communication with a display e.g. computer screen, which is configured to compare a digital representation, which is derivable from the representation captured by the camera, of the 3D structure present at the far end of the tube, to pre-stored data regarding 3D structures of each of plural regions (of the human body e.g. of one or more vertebrae), to select a “most similar” region, from among the plural regions, whose 3D structure is most similar to the digital representation, and to determine what the “most similar” region's orientation was, relative to a standard (e.g. coordinate system e.g. relative to the orientation of the tube), when said camera captured said camera-captured representation of the 3D structure, typically using pre-stored data regarding 3D structures of each of plural regions (of the human body e.g. of one or more vertebrae) to generate an image of regions adjacent to the “most similar” region, wherein the regions are shown at an orientation which equals the “most similar” region's orientation. Typically, the representation as captured comprises a range image of the 3D structure which is present at a far end of a tube, and the camera generates from the range image, a 3D representation of the 3D structure present at the far end of the tube.

Certain embodiments seek to use surface matching to match a surface measured by a 3D camera during surgery, to a bone surface extracted from a pre-operative image, such as but not limited to a CT image.

Certain embodiments seek to provide software which computes coordinate transformation (3D rotation and/or 3D translation) between vertebra and a tube and between the tube and the coordinate system of a 3D camera (3DC) and to generate, accordingly, a display of vertebra and tool tip, wherein the tool is tracked to ensure that the current position of the tool tip is always being displayed to the surgeon.

Certain embodiments seek to provide a method in which an imager provides pre-operative imagery e.g. CT images; and/or a hardware processor derives a 3D image from the pre-operative images; and/or a surgeon marks a surgical field on the pre-operative images or on the 3D image derived therefrom and/or the hardware processor marks the surgical field identified by the surgeon, in the 3D image and/or the hardware processor tracks the surgical tool's relative position and orientation and/or the hardware processor converts relative position and orientation to actual position, and superimposes the tool onto the 3D image accordingly.

Certain embodiments seek to provide a system which is configured for direct tracking of regions of exposed vertebrae, or other exposed bone areas, and also for direct tracking of surgical tools, e.g. via markers attached to the tools.

Certain embodiments seek to provide a system which is configured to assign ‘global’ or ‘world’ or absolute (rather than merely relative) coordinates to a 3D image of a surgical field imaged by a miniature camera.

It is appreciated that any reference herein to, or recitation of, a stage or an operation being performed, e.g. if the operation is performed at least partly in software, is intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by, server A. Analogously, the remote processor P may not, itself, perform all of the operations, and, instead, the remote processor P itself may receive output/s of portion/s of the operation from yet another processor/s P′, may be deployed off-shore relative to P, or “on a cloud”, and so forth.

There is thus provided, in accordance with at least one embodiment of the present invention,

The present invention typically includes at least the following embodiments:

Embodiment 1. An imaging system aka 3d camera operative in conjunction with a tube (e.g. retractor or trocar) having two open ends, the system comprising:

active portions small enough to fit into the tube; and/or

an electronic subsystem including a hardware processor operative to receive at least one image from said active portions and to generate therefrom at least one 3D image of a scene (aka miniature scene) visible via one of the tube's open ends (aka portion of a surgical field aka topology).

The tube may be a surgical tube (e.g. retractor or trocar). The scene may refer to whatever is visible via one end of an open tube, which may be inserted into the human body e.g. in order to image one or more exposed vertebrae, where the 3D camera is secured to the other open end of the open tube. The inner surface of the tube's walls may be opaque e.g. black coated.

The camera may be secured to and disengaged from the tube e.g. retractor, more than once, even during a single surgical procedure. For example,

a. During a surgical process such as a bone removal process the M3D (or miniature) camera can be entered into the tube multiple times to measure progress of the surgical process and provide feedback to the surgeon regarding, say, the amount of bone removed (in 3D) and/or how close the final bone is to the pre-operational design goal. The M3D camera can be inserted into the tube each time the surgeon seeks to determine, measure or view how close s/he is to a desired end result or goal. This may be done multiple times, e.g. if the surgeon is approaching the end result only by very small increments (e.g. is removing bone in several rounds, only a little bit in each round) e.g. due to extreme adjacency of the bone to a sensitive body part such as a nerve or blood vessel, and/or

b. the M3D camera can be inserted into the tube each time the surgeon changes the orientation and/or position of the retractor e.g. in order to verify the retractor's new position/orientation vs. vertebra.

Embodiment 2. The system of any of the preceding embodiments and also comprising a tracker configured to be secured to the tube, thereby to monitor an absolute location of the retractor.

It is appreciated that any suitable surgical tool tracker may be used such as:

a. the tracker described in the co-owned PCT application, which tracks markers attached to surgical tools, thereby to yield 3D position and orientation of the tool itself; and

b. ndigital.com's Polaris family of products, also operative to track the 3D position and orientation of active or passive markers attached to surgical tools.

The tracker is typically used in conjunction with the markers and miniature camera described herein.

Typically, each tracker or tracking unit herein includes all or any subset of: an Inertial Measurement Unit (IMU), wireless communication module, indicator marks for user feedback, and fiducial/ball markers for tool tracking. Each tracker or tracking unit typically measures and reports its own (and that of any tool or tube secured thereto) position and orientation in real time, typically via wireless communication.

The term “tracking”, “tracker” and similar as used herein is intended to include any conventional method for tracking position and/or orientation (e.g. pitch and/or yaw and/or roll) of a display and/or of devices. Sensors may be used to continuously record signals from transmitters adjacent (or aboard) tracked objects. The signals may be used to estimate objects' physical locations. Any suitable coordinate system may be employed e.g. Cartesian, polar, and the cylindrical system. Inertial tracking use data from accelerometers which measure linear acceleration and/or gyroscopes which measure angular velocity used for rotational tracking. Inertial Measurement Units systems (IMU), based on MEMS components, facilitate high update rates and reduce latency. Inertial sensors are configured for tracking both rotational and translational movement.

Embodiment 3. The system of any of the preceding embodiments and wherein said active portions comprise:
at least one image sensor/s or cameras oriented to have a partially or totally overlapping field of view, and
at least one structured light projector/s projecting a known pattern onto the field of view of the image sensor/s.

Structured light projectors are configured to project a known pattern on to a scene. the way the pattern deforms when striking surfaces, such as a topology visible via an open tube (tube with 2 open ends), allows the depth and surface information of the topology or objects or features visible via the tube, to be determined or computed.

Embodiment 4. The system of any of the preceding embodiments and wherein at least one dimension of said active portions is smaller than the tube's inner diameter.

Embodiment 5. The system of any of the preceding embodiments which includes at least one component which is larger in size than the tube's inner diameter.

Embodiment 6. The system of any of the preceding embodiments and wherein the imaging system includes at least one mechanical subsystem configured to secure the camera at a fixed location and orientation vs. markers that track the tube.

Embodiment 7. The system of any of the preceding embodiments and wherein the hardware processor receives data from the at least one image sensor and generates said 3D image from said data.

Embodiment 8. The system of any of the preceding embodiments and wherein at least one image sensor is deployed at an offset from at least one structured light projector and wherein the offset is known to the hardware processor and is used for triangulation which generates said 3D image from said data.

Embodiment 9. The system of any of the preceding embodiments and wherein the hardware processor assigns absolute coordinates to the 3d image of the surgical field.

Embodiment 10. The system of any of the preceding embodiments and wherein the hardware processor is also configured to monitor a tool which is tracked, hence its absolute coordinates are known, and is moving (e.g. in order to perform a surgical operation on a portion of a vertebra).

The section or portion of the vertebra on which the surgical operation is performed may differ from the section or portion of the vertebra which is exposed, hence visible. For example, the tubular retractor may be inserted on the left side of the spine, in order to register the vertebra, and then, a tracked screwdriver may be guided or monitored to place a pedicle screw on the right side of the same vertebra.

Embodiment 11. The system of any of the preceding embodiments and wherein the hardware processor is configured to recognize a location of the scene within a larger topology e.g. a 3D representation of one or more vertebrae.

Embodiment 12. The system of any of the preceding embodiments and wherein said tool is deployed inside the retractor.

Embodiment 13. The system of any of the preceding embodiments and wherein said tool is deployed outside the retractor.

Embodiment 14. The system of any of the preceding embodiments and wherein at least one pre-operative image, having a resolution, represents the larger pre-mapped topology, and wherein the 3d camera have a resolution which is of the same order of magnitude as the pre-operative image's resolution, thereby to yield a cost-effective system.

Embodiment 15. The system of any of the preceding embodiments and wherein at least one pre-operative image, having a resolution, represents the larger pre-mapped topology, and wherein the 3d camera have a resolution which is at most one order of magnitude more accurate than the pre-operative image's resolution, thereby to yield a cost-effective system.

Embodiment 16. The system of any of the preceding embodiments wherein the mechanical subsystem is larger in size than the tube's inner diameter.

Embodiment 17. The system of any of the preceding embodiments and wherein said active portions also comprise LED light and optics.

Embodiment 18. The system of any of the preceding embodiments wherein said at least one image sensors comprises two image sensors, and wherein said triangulation comprises stereo triangulation.

It is appreciated that stereo triangulation, which is available as a MatLab function inter alia, reconstructs 3D points from projections thereof in two images of a scene e.g. the topography visible via a tube.

Embodiment 19. The system of any of the preceding embodiments wherein said at least one projector comprises but a single projector, said at least one image sensor comprises but a single image sensor, and wherein pattern correlation and measurement of sub-pattern displacement are used for said triangulation or for depth estimation.

Embodiment 20. The system of any of the preceding embodiments and wherein the pre-operative image comprises a CT image.

Embodiment 21. An imaging method operative in conjunction with a tube having two open ends, the method comprising:

providing a 3d camera with active portions small enough to fit into the tube and

using an electronic subsystem including a hardware processor operative to receive at least one image from said active portions and to generate therefrom at least one 3D image of a scene.

Embodiment 22. The method of any of the preceding embodiments and wherein the tube bears fiducial markers and wherein the tube's location in space is known to said hardware processor due to said markers.

Embodiment 23. The method of any of the preceding embodiments and wherein at least when an inferior edge of at least one lamina and/or ipsilateral base of spinous process are identified, the 3d camera is secured to a top end of the tube, and the inferior edge of the lamina, as viewed through the bottom end of the tube, is measured, thereby to yield a measured surface; and wherein at least one vertebra's 3D location is presented to a human user, thereby to facilitate performance of Tubular Laminotomy.

Embodiment 24. The method of any of the preceding embodiments wherein said vertebra's 3D location is derived by matching the measured surface to a portion of a 3D image of at least a portion of the lamina and from the tube's known location in space.

Embodiment 25. The method of any of the preceding embodiments wherein the camera is secured to a top end of the tube and measures an inferior articulating facet, as viewed through the bottom end of the tube, and wherein at least one vertebra's 3D location is presented to a human user, thereby to facilitate performance of MIS TLIF (Transforaminal Interbody Fusion).

Embodiment 26. The method of any of the preceding embodiments, said vertebra's 3D location being derived from a 3D image of the facet and from the tube's known location in space.

Embodiment 27. The system of any of the preceding embodiments and also comprising at least one tool tracker and wherein the hardware processor is configured to use data from the tracker to be presented to a human user, thereby enabling the human user to monitor a current position of the tool.

Embodiment 28. The system of any of the preceding embodiments and also comprising at least one tracker attached to the tube, and wherein the hardware processor is configured to use data from the tracker to be presented to a human user, thereby enabling the human user to monitor a current position of the tube.

Embodiment 29. The system of any of the preceding embodiments and wherein the hardware processor is configured to superimpose the 3D image of the miniature scene onto an earlier captured image of a larger scene which is larger than, and includes, the miniature scene, thereby to generate a superimposed image, and to display the superimposed image to a human user.

Embodiment 30. A computer program product, comprising a non-transitory tangible computer readable medium having computer readable program code embodied therein, said computer readable program code adapted to be executed to implement An imaging method operative in conjunction with a tube having two open ends, the method comprising:

receiving at least one image from active portions, of a 3d camera, which are small enough to fit into the tube and

using a hardware processor to generate therefrom at least one 3D image of a scene.

The present invention also typically includes at least the following embodiments:

Embodiment b1. An improved MISS method comprising:

Providing equipment, other than radiation equipment, which enhances visual orientation of a surgeon whose unaided view of the spine and internal structure is reduced due to the limited open areas available to the surgeon in MISS.

Embodiment b2. The method according to any of the preceding embodiments wherein the equipment comprises navigation equipment which provides the surgeon with guidance for accurate placement of implants.

Embodiment b3. The method according to any of the preceding embodiments wherein the equipment is operative for optical scanning and tracking of spine vertebrae bone surface.

Embodiment b4. The method according to any of the preceding embodiments wherein the equipment is operative for ultrasound scanning and tracking of spine vertebrae bone surface.

Embodiment b5. An improved MISS navigation system operative in conjunction with all or any subset of: a 3D camera, at least one tool tracking unit/s, surgery tools, a MISS retractor having an internal tube, and a computer in data communication with the 3D camera and tool tracking unit/s, the system comprising:

at least one camera, aka Miniature 3D camera/s or M3D, attached to the retractor/s inside tube.

Embodiment b6. The system according to any of the preceding embodiments and also comprising a ultrasound transceiver and a tool tracking unit attached to the transceiver.

Embodiment b7. The system according to any of the preceding embodiments and also comprising a 3D camera which tracks at least the ultrasound transceiver.

Embodiment b8. The system according to any of the preceding embodiments and also comprising a 3D camera which tracks at least the surgery tools, the MISS retractor and the ultrasound transceiver.

Embodiment b9. The system according to any of the preceding embodiments and also comprising a 3D camera which tracks at least the surgery tools and the MISS retractor.

Embodiment b10. The system according to any of the preceding embodiments and also comprising a tool tracking unit that includes all or any subset of: an Inertial Measurement Unit (IMU), wireless communication module, indicator marks for user feedback, and fiducial/ball markers for tool tracking.

Embodiment b11. Processing circuitry comprising at least one processor and at least one memory and configured to perform at least one of or any combination of the described stages or to execute any combination of the described modules.

Also provided, excluding signals, is a computer program comprising computer program code means for performing any of the methods shown and described herein when said program is run on at least one computer; and a computer program product, comprising a typically non-transitory computer-usable or -readable medium e.g. non-transitory computer-usable or -readable storage medium, typically tangible, having a computer readable program code embodied therein, said computer readable program code adapted to be executed to implement any or all of the methods shown and described herein. The operations in accordance with the teachings herein may be performed by at least one computer specially constructed for the desired purposes or general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations or stages within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities e.g. within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing system, communication devices, processors (e.g. digital signal processor (DSP), microcontrollers, field programmable gate array (FPGA), application specific integrated circuit (ASIC), etc.) and other electronic computing devices. Any reference to a computer, controller or processor is intended to include one or more hardware devices e.g. chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components and, alternatively, may be the same structure. A statement that an element or feature may exist is intended to include (a) embodiments in which the element or feature exists; (b) embodiments in which the element or feature does not exist; and (c) embodiments in which the element or feature exist selectably e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein, and/or to perform functionalities described herein and/or to implement any engine, interface or other system illustrated or described herein. Any suitable computerized data storage, e.g. computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein which may, for example include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus the term user interface or “UI” as used herein, includes also the underlying logic which controls the data presented to the user e.g. by the system display and receives and processes and/or provides to other modules herein, data entered by a user e.g. using her or his workstation/device.

### BRIEF DESCRIPTION OF THE DRAWINGS

Example embodiments are illustrated in the various drawings. Specifically:

FIGS. 1-4 are schematic diagrams of certain embodiments which may be provided standalone or in any suitable combination;

FIGS. 5a-6 are simplified flowchart illustrations of methods provided in accordance with certain embodiments e.g. in conjunction with any of the embodiments of FIGS. 1-4. Each method typically comprises all or any subset of the illustrated stages, suitably ordered e.g. as shown.

Certain embodiments of the present invention are illustrated in the following drawings; in the block diagrams, arrows between modules may be implemented as APIs and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML.

Methods and systems included in the scope of the present invention may include any subset or all of the functional blocks shown in the specifically illustrated implementations by way of example, in any suitable order e.g. as shown. Flows may include all or any subset of the illustrated stages, suitably ordered e.g. as shown. Tables herein may include all or any subset of the fields and/or records and/or cells and/or rows and/or columns described.

Computational, functional or logical components described and illustrated herein can be implemented in various forms, for example, as hardware circuits such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences such as but not limited to objects, procedures, functions, routines and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the stages included in such methods, or in accordance with methods known in the art.

Any logical functionality described herein may be implemented as a real time application, if and as appropriate, and which may employ any suitable architectural option, such as but not limited to FPGA, ASIC or DSP or any suitable combination thereof.

Any hardware component mentioned herein may in fact include either one or more hardware devices e.g. chips, which may be co-located or remote from one another.

Any method described herein is intended to include within the scope of the embodiments of the present invention also any software or computer program performing all or any subset of the method's stages, including a mobile application, platform or operating system e.g. as stored in a medium, as well as combining the computer program with a hardware device to perform all or any subset of the stages of the method, suitably ordered e.g. as described herein.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use; and which is based on any suitable technologies such as semiconductor, magnetic, optical, paper and others.

### DETAILED DESCRIPTION OF CERTAIN EMBODIMENTS

Conventional MISS reduces the surgeon's visual orientation by limiting the actual open areas to relatively small (about 25 mm diameter) openings, showing limited view of the spine and internal structure. In procedures where the surgeon elects to use only a single incision—for insertion of two screws, rods, and cage, while placing the other screws percutaneously—the surgeon may require guidance for accurate placement of the implants. Often this means additional radiation for the patient and sometimes the surgeon—either by CT (full or partial), or fluoroscopy.

There is a growing need for a navigation system that allows accurate information flow or guidance and implant placement without added radiation to either staff or patient. Certain embodiments herein answer this need e.g. by optical and ultrasound scanning and/or tracking of spine vertebrae bone surface.

The MISS navigation system includes, according to certain embodiments, all or any subset of the following hardware components:
1. 3D camera aka 3DC that tracks the surgery tools, MISS retractor, and optional ultrasound transceiver
2. Surgery Tool tracking unit/s aka STT that each include an Inertial Measurement Unit (IMU), wireless communication module, indicator marks for user feedback, and fiducial/sphere markers for tool tracking
3. A computer that runs the surgery software and connects to both the 3D camera aka 3DC and tool tracking units by either wired or wireless communications
4. At least one miniature 3D (M3D) camera/s attached to retractor/s inside tubes
5. Tool tracking unit/s attached to MISS retractor tube/s for tracking by 3D camera aka 3DC
6. Optional ultrasound transceiver including an attached tool tracking unit secured thereto.

Retractor sets as available e.g. from medikrebsusa.com, typically include several retractors of different diameters, and/or or different lengths (ranging say from 4 cm to 7 cm); these various lengths may be used selectably or selectively by a surgeon e.g. depending on the thickness of the fat layer that may separate vertebrae from the skin and outer world. Retractor lengths may vary, say, from 30 mm to 90 mm in, say, 10 mm intervals. External diameters may vary, say, from 12 mm to 26 mm in 2 mm intervals. For example, medfix.com markets a retractor set which includes 4 lengths (50, 60, 70, 80 mm)×3 diameters (18, 22, 26 mm)—a total of 12 tubes.

According to some embodiments, a group of cameras is provided, including one camera per retractor diameter, since typically, retractor sets include plural retractors with plural different diameters respectively.

According to other embodiments, a single camera is provided, which is smaller than the smallest expected or common tube diameter (e.g., the single camera's active parts may have a dimension which is less than 12 mm if the narrowest retractor in a retractor set is known to have a tube whose external-internal diameters are 14 mm-12 mm respectively.

According to some embodiments, adapters are provided to allow a single camera to be used for tubes of several different lengths and/or of several different diameters, all larger than the dimension of the single camera's active parts. For example, the M3D camera described in Example 2 herein, is 12 mm in diameter. This fits well in a 14 mm external/12 mm internal diameter tube. However, for use with a 26 mm external/24 mm internal tube, for example, an adapter may be provided, such as a ring with an internal diameter of 12 mm and external diameter of 24 mm.

The system described in co-owned PCT patent application PCT/IL2019/050775, incorporated herein by reference, is an example of a system which provides the first three components above: 3D camera, tool tracking units, and computer. The additional M3D camera/s (4) and tool tracking units attached to MISS retractor tube/s (5) add the capability to ‘see’ bone structure through relatively narrow openings common in MISS, and to track MISS tubes during surgery.

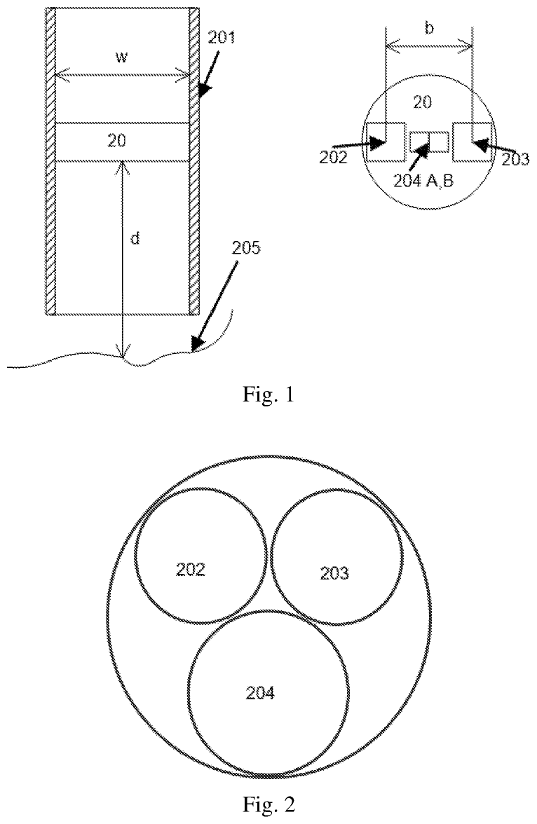

Typically, each M3D camera secured to a tube, having range imaging functionality, provides accurate 3D measurement of a bone section exposed by the surgeon through the opening or via the tube. FIG. 1 shows an example of a M3D camera secured to a tube e.g. retractor, or inside a retractor. The retractor location is measured by the 3D camera, aka 3DC, and in this way the location of the bone section is registered to the 3D camera aka 3DC coordinate system. The information provided by the M3D camera can be placed in or aligned to (e.g. by rigid coordination transformation) the ‘external’ (aka 3D camera or ‘world’) coordinate system measured by the 3D camera.

Typically, the surgeon marks the exposed area on the pre-operational CT and the bone structure is registered to the bone structure derived from the CT. The output of the registration stage is a set of rigid transformations relating each vertebra in the pre-operational CT to the vertebra's location and orientation as measured by the 3DC and M3D cameras in the OR (operating room) setting.

In case of multiple incisions with multiple retractors, the registration over multiple areas of the same vertebra allows accurate registration of the vertebra position to the CT. However, some MISS procedures use a single incision per vertebra. In this case one needs to ensure enough bone exposure, visible through the single incision, to allow good registration of the full vertebra. See detailed description for example of ‘enough’ bone surface.

Since the openings in MISS are small and the exposed bone area is limited, additional information that can be integrated with the M3D and 3DC cameras information may be used. The additional information may be obtained from existing navigation solutions such as markers attached to bone structures and/or intraoperative CT scan, fluoroscopy with markers, etc. In certain embodiments, the additional information is obtained from accurate tracking of an ultrasound probe location and angle with the 3D camera aka 3DC. Matching the bone surface structure in the ultrasound image with the bone surface derived from the preoperational imagery e.g. CT, provides an ‘internal’ view of the vertebrae positions that enhances the system's accuracy.

The M3D camera is configured and operative for use in MISS surgery including being secured onto the retractor and viewing vertebrae via the retractor opening. For example, the camera system (e.g. as shown in FIG. 1, reference numeral 20) may include all or any subset of a structured light projector 204 (optionally built from 2 light projector units A and B—see example I) and two video cameras 202 and 203 typically placed on either side of the projector 204 and integrated in a single housing to form a single camera unit 20. As is well known in the art, the design may include only a single camera 202 or 203: (https://en.wikipedia.org/wiki/Structured-light_3D_scanner). The projector 204 Field of View (FOV) and the cameras 202 and 203 FOV are aligned to include the same area on the bone surface 205, preferably having an FOV slightly larger than tube 201 diameter. The M3D camera is shown in cross-section typically inserted in or secured to sample cylindrical retractor tube 201 at a distance from the bone surface 205 that equals the working distance of the camera—e.g. see example I described below.

FIG. 1 shows a cross-sectional side view of the retractor tube into which an M3D camera has been inserted on the left, and a bottom view (side facing bone surface 205) of the camera unit 20 on the right. It is appreciated that “insertion” as used herein may refer to partial insertion e.g. in which only the active portions of the camera are inserted into or secured to the tube to enable them to view the bottom end of the tube.

The structured light projector 204 typically projects light towards bone surface 205, the projection forming a known pattern that illuminates bone surface 205. The pattern may be a random dot pattern, lines, binary coded images, or any of other patterns known in the art. The two cameras 202 and 203 determine the 3D shape of bone surface 205 by comparing the pixel coordinates of the detected illumination pattern between the two cameras. The co-owned PCT application describes an example method for 3D distance computation from pixel offsets.

### EXAMPLE I

Providing an off-the-shelf example for low-cost implementation, units A, B may each comprise an off-the-shelf miniature structured light projector e.g. AMS BELICE-850 dot pattern illuminator for 3D stereoscopic imaging (www.ams.com). The single projector package size may be 3.4 mm×3.5 mm×3.56 mm. the projector may produce 5500 dots in a camera field of view (FOV) of 68°×48°. The configuration typically includes two adjacent projectors—type A and B—placed 4.0 mm apart, where type A and B produce a rectangular pattern of points at 5° and 15° inclination to the line connecting the projectors, respectively.

An example of suitable off-the-shelf miniature devices to implement video cameras 202, 203 (which may be deployed parallel to one another), are Misumi MD-BL24M7 B/W camera, 640×480 pixels, f=1.83 mm lens, F#=5.0 (Numerical aperture), 7 mm width, optional IR LED and IR pass filter (www.misumi.com.tw). The camera minimal viewing angle is 56°×44° so the example projector FOV of 68°×48° is slightly larger than the camera FOV, ensuring the full camera FOV is covered by the projected patterns. The camera PCB can be changed to allow integration of two cameras and two projector units on one PCB, where the two cameras are placed on both sides of the projector, e.g. as shown in FIG. 1.

The baseline distance b between the cameras 202, 203 and the projection angle of the projector 204 may be specified to match, say, a required FOV and distance d from bone surface 205 while allowing insertion into the retractor tube 201. Taking a retractor tube inside diameter of w=20 mm, for example, and circular FOV of θ=44°, puts the M3D camera at a distance of d=cotan(θ/2)*w/2=24.8 mm from bone surface 205. The depth of field (DOF) of the cameras 202, 203 may be >5 mm to allow for varying distance to the features of bone surface 205.

Typically, DOF≈2*d²*F#*c/f² (https://en.wikipedia.org/wiki/Depth_of_field) where d is the distance between cameras 202, 203 and bone surface 205, F# is the numerical aperture, c circle of confusion (typically the pixel size for the camera) and f the focal length. Taking the data above −d=25 mm, F#=5, c=0.003 mm (3 micron pixel), f=1.83 mm, we get DOF=5.6 mm.

The projector 204 may send, say, about 5000 points on a rectangular FOV, or about 60 points across for a circular FOV, giving spatial sampling with resolution of 0.33 mm for 20 mm diameter FOV. The depth resolution may depend on the offset between the images of the same projected dot between the two cameras 202, 203. For example, taking a baseline distance between image sensor centers b=14 mm, the offset between images of the same dot for bone surface 205 distance d=25 mm and focal length f=1.83 mm, parallel cameras, may be offset Δ=0.51 mm. The geometry gives δ d/d=δΔ/Δ, so changing the offset by two pixels (one for each camera) δΔ=0.006 mm, may yield δd=0.3 mm as the depth resolution of this example setup.

The parameters of the camera, projector, and their optics are given as examples only. Example I demonstrates that even off-the-shelf components can yield a setup satisfying the available space and restrictions of MISS surgery so as to provide a system that can give 3D information having the required accuracy over the full available FOV.

The projector and cameras may be characterized by all or any subset of the following:
1. Spatial resolution and depth resolution equal the required system resolution, typically 0.3 mm
2. Projector and cameras FOV at exit of tube are slightly larger than the internal diameter of the retractor tube
3. Camera DOF has larger than expected variation in depth of bone surface, typically >5 mm
4. Distance of M3D camera from bone surface is determined according to FOV, DOF, resolution

In the “basic” configuration described in FIG. 1, the cameras 202, 203 are equipped with simple single focus imaging lenses. Since the use of the M3D camera 20 as a medical device requires sterilization, the basic configuration of FIG. 1 allows for a solution so cost effective that the unit may even be used on a single use, disposable basis.

It is, however, also possible to use optics configured to achieve higher resolution depth images e.g. all or any subset of the following:
1. Variable focus camera lens—typically allows higher resolution by requiring a smaller depth of field and adjusting the focal length of the lens to ‘scan’ the various distances from the object. An example of an off-the-shelf variable lens is Corning Varioptics A16F lens (www.corning.com) which has a 20 diopters change of focus and the lens' small 6.2 mm diameter is typically compatible with embodiments herein.
2. Building endoscopic ‘optical wave guides’ that typically allow wider angular separation between the images of the two cameras and consequentially a wider effective baseline distance between them. For example, the setup described in Example I includes baseline distance b=14 mm and object distance d=25 mm, giving an effective baseline angle (angle between rays collected by the two cameras) of beta=2*atan(b/2d)=16°. Placing folding mirrors or prisms that collect light at a relative angle of 32° will provide X2 better depth resolution. The wider baseline can be achieved either by placing the folding optics utilizing the full internal width of the retractor, by placing the M3D setup closer to the bone surface, or both.
3. Fiber optic design—using fiber bundles for structured light and/or image collection. The fiber bundles may be integrated into the retractor side walls and allow minimum interference to placing tools through the retractor while operating the M3D camera.

Options 1-3 described above—separately or in any combination—can improve the M3D camera performance at the expense of added complexity and/or cost. The M3D design is not limited to the examples above and can utilize zoom and/or telephoto lenses and/or flexible image guiding optics, or other camera options known in the art.

The sizes listed in Example 1 are example sizes which may be used in MISS retractors. However, the design is not limited to these sizes. Some MISS procedures use endoscopic tube entry ports—narrow tubes that are typically 8 mm in diameter and support use of endoscopic tools and vision systems. In this case, the M3D camera design may change to fit the smaller tube diameter. https://www.sciencedirect.com/science/article/abs/pii/S1361841512000473?via%3Dihub) aka Reference 2, describes an endoscopic 3D scanner based on structured light, having a diameter of 3.6 mm. Other optical components (camera/s, scanner) and designs may be used to allow fitting the M3D camera in a more confined tube.

### Example 2

Example 2, also based on off-the-shelf hardware, is a design with a customized laser projector that allows the M3D camera 20 to fit in smaller retractor tubes, such as a 14 mm external diameter/12 mm internal diameter retractor tube. Using as cameras 202 and 203, for example, Missumi MD-B311M5-77 B/W miniature camera, 4.9 mm diameter, 1920×1080 pixels, 1.4×1.4 um pixel size, f=1.83 mm lens, with distance b between camera centers b=5 mm (the two cameras adjacent to each other), and as laser projector 204 a customized 6 mm diameter projector using a single mode VCSEL light source (for example, II-VI APA8501010001, https://www.ii-vi.com/product/850 nm-low-power-sm-chip/?sfw=pass1603358166), focusing lens f=6 mm, diameter=6 mm (for example Edmund Optics 36-626 https://www.edmundoptics.com/p/6 mm-dia-x-6 mm-f1-small-diameter-plastic-aspheric-lens/34651/), and random-dot diffractive optics pattern generator (for example, Holoor MS-469-850-N-X, https://www.holoor.co.il/structured-light-doe/) diced to 4 mm×4 mm square, so the diagonal is 5.6 mm.

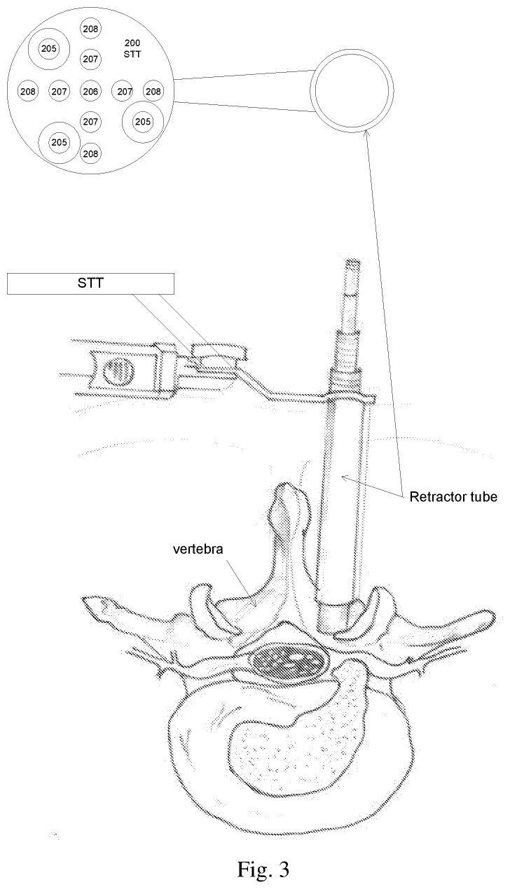

The cross section of the M3D camera in this example is shown in FIG. 2 and shows the external diameter is 12 mm, fitting in a retractor with external diameter of 14 mm having a 1 mm width tube. In addition to the two cameras 202, 203 and projector 204 the M3D camera may include LEDs—single or multiple—to provide non-patterned illumination of the target scene.

Both Example 1 and Example 2 are only examples of a miniature 3D camera which may be used in MISS operations according to certain embodiments herein.

The term “miniature” is used herein to include any camera whose active portions are small enough to fit into a surgical tube e.g. one or more dimensions of all or any subset of the two image sensors, projector, LED light, and their optics are typically smaller than the tube's inner diameter. The remaining portions of the camera e.g. electronics and/or mechanics may be larger in size than the tube's inner diameter. Typically, methods described in the co-owned PCT patent application are used to assign ‘global’ or ‘world’ or absolute (rather than merely relative) coordinates to the 3D image of the surgical field imaged by the miniature camera.

According to certain embodiments, the Surgery Tool Tracking unit or tracker is secured or attached to the MISS retractor tube. The M3D camera enables 3D view and registration of spine vertebrae bone features through the MISS retractor tube/s. To register the 3D structure measured by the M3D camera to the coordinate system of the 3D camera aka 3DC, the M3D camera location and orientation need to be measured by the 3D camera aka 3DC. For this purpose, each retractor tube has a tracker or Surgery Tool Tracking unit aka STT attached thereto. A detailed description of an example STT and example methods for use of an STT are provided in PCT/IL2019/050775. The 3D camera aka 3DC tracks the fiducials/markers on the STT or tracker, enabling transfer of the 3D coordinates from M3D to 3DC coordinates. Alternatively or in addition, the IMU that is part of each STT unit or tracker provides added information on the tube orientation, and can be used to track and alert tube motion during the surgery. Alternatively or in addition, the feedback LEDs on each STT or tracker may also be used to align the tube to a desired angle, in a similar way that the STT or tracker is used to align surgery tools to the planned angle, e.g. as described in PCT/IL2019/050775.

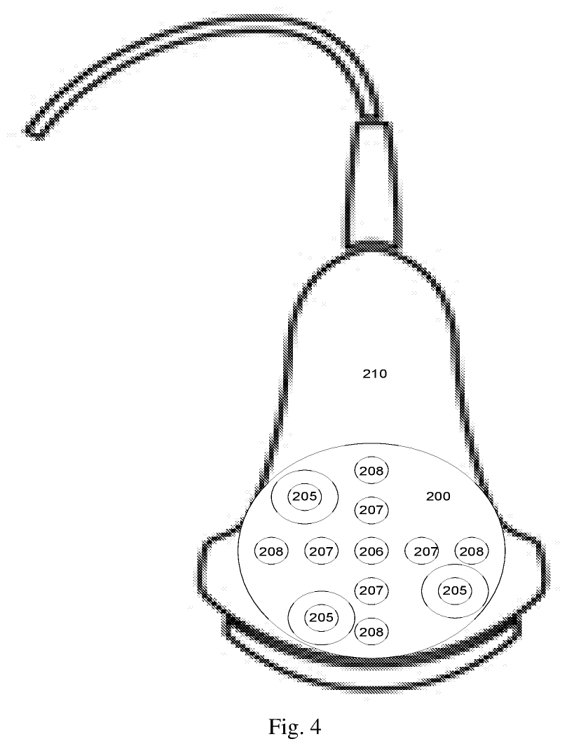

FIG. 3 shows a MISS retractor tube with a STT unit secured thereto which allows tracking of the STT unit or tracker by the 3D camera aka 3DC and does not disturb free access to the retractor tube by the surgeon. The combination of M3D and STT in each retractor tube provides registration which then allows continuous tracking of the spine vertebrae during MISS procedures.

Specifically, FIG. 3 is a schematic top view and side-view diagram of an STT unit secured to an MISS retractor tube; as shown in the side view at the bottom of the drawings, the tube may be inserted into an incision to provide the surgeon with access to at least one vertebra. The Surgery Tool Tracking Unit or tracker may include both passive markers/fiducials 205, and active LEDs 206-208 or may include only the passive markers 205—for example, a passive Dynamic Reference Frame (DRF) with reflecting ball markers (at least 3) that allows tracking by a 3D camera aka 3DC of the Dynamic Reference Frame position and orientation.

System operation according to an embodiment of the invention is now described. The M3D camera integrates with the rest of the system components to allow registration of a specific bone feature on a single vertebra to the pre-operation CT. An example MISS sequence, aka fow1, may include all or any subset of the following stages, suitably ordered e.g. as follows:
1. Surgeon makes first incision and inserts MISS retractor into the incision. The retractor is locked in position, for example by the surgeon activating a mechanical lock mechanism (see https://www.aesculapimplantsystems.com/products/spine-solutions/thoracolumbar-solutions/spyder-mis-retractor-system)
2. Surgeon exposes and clears a section of bone including identifiable features through the retractor tube
3. Surgeon inserts M3D camera into retractor tube and locks the camera in position
4. Surgeon marks the exposed bone feature on pre-operation CT using system software which may include all or any subset of the software functionalities described in the co-owned PCT application which is hereby incorporated by reference.
5. Software performs registration of vertebra—measure bone feature 3D shape from M3D camera typically combined with 3D position of retractor tube from 3D camera aka 3DC
6. Software presents to surgeon the retractor superimposed on CT image typically with successful registration indication
7. Surgeon takes out M3D camera from retractor tube to clear tube for other surgery tools (or the M3D design may use minimal cross section and may be kept inside retractor tube during surgery)
8. Surgeon shows surgery tools to 3D camera aka 3DC for tool registration and tracking
9. Surgeon verifies vertebra registration e.g. by placing tool tip on known bone feature and seeing the software present the tool tip at the known bone feature on monitor showing tool and CT
10. Surgeon repeats stages 1-6 and 9 for registering each vertebra using all existing retractors
11. Software and system continuously track the positions and angles of retractors and tools using 3D camera aka 3DC and markers e.g. the STT on the tubes e.g. retractors and/or DRFs on tools, e.g. as described in our PCT
12. Software alerts surgeon to any changes in retractor positions that may affect registration, e.g. detected motion >2 mm (or other chosen threshold)
13. Surgeon repeats stages 3-6 and 9 for any retractor with motion alert to verify proper registration

The registration accuracy of a bone features from CT to 3D camera aka 3DC typically depends on the CT slice thickness (typically 1 mm for high density scan), 3D camera aka 3DC resolution (typically 0.3 mm), exposed area size (typically 10 mm×10 mm) and bone contrast in CT image. From past trials and from literature—see (https://link.springer.com/article/10.1007%2Fs00586-004-0797-y; aka reference 3—the expected registration error for proper choice of features and points is 0.5 mm.

Taking a 0.5 mm error over an exposed bone area of 10 mm gives angular error (in two angular directions) of 80=arctan(0.5/10)=3°. For single incision on one side of a vertebra, taking a typical distance of 25 mm between the two pedicles of the same vertebra, yields an error of 1.25 mm for the counter-side pedicle location in addition to 3° angular error in pedicle screw direction. This makes the single incision registration usable also for the counter-side pedicle without additional information, enhancing the overall registration accuracy.

Use of M3D camera and two incisions per vertebra—preferably on both left and right sides of the same vertebra—provides enhanced accuracy in the registration of each vertebra. The positional accuracy can be kept close to the system's resolution at around 0.3 mm.

Following the registration stage 6 the system 3D camera aka 3DC may be used for tracking of tools and retractor positions continuously during the operation. The M3D camera can be re-inserted into or re-secured to each retractor at any time during the surgery to verify the registration accuracy e.g. as described herein with reference to stages 12, 13.

Continuous tracking of each vertebra is also possible using the M3D camera, with a design that includes minimum interference with the use of the retractors as entry points for the surgery. The ‘endoscopic’ or fiber-optic design options described above may be used for leaving large usable internal space in the retractor and/or for allowing continuous vertebra tracking.

Preferred methods for executing two conventional MISS surgeries are now described by way of example; the M3D camera is used during the surgeries and enhances the surgeon's performance.

Step by step images of conventional MISS are available on the AOSpine website (https://aospine.aofoundation.org/education).

### MISS Example 1: Tubular Laminotomy

According to certain embodiments, this surgery may include all or any subset of the following stages, suitably ordered e.g. as shown:
1. Tube placement for L4 laminotomy—the tube typically carries, bears, includes or is marked with a set of at least one fiducial markers that are identified by the system 3D camera aka 3DC and located in space. These markers may include any feature, marking or object, typically of known pattern and/or size, that is deployed in a camera's field of view and is then used as a point of reference or measure.
2. Removal of soft tissue and identification of inferior edge of the lamina and ipsilateral base of spinous process—in this stage, following tissue removal, the M3D camera is inserted into or secured to the tube and measures the inferior edge of the lamina. Using the 3D image of the lamina edge and the location of the tube in space allows locating the full vertebra (in this case L4) in 3D space, and directs the surgeon to adjust the tube if the relative vertebra location requires adjustment.
3. Laminotomy—drilling of the medial portion of the lamina in a cranial direction and bone removal. During a surgical process e.g. the bone removal process the M3D camera can be entered into the tube multiple times e.g. to measure the bone removal process and provide feedback to the surgeon regarding the amount of bone removed (in 3D) and how close the final bone is to the pre-operational design goal.

### MISS Example 2: MIS TLIF (Transforaminal Interbody Fusion)

According to certain embodiments, this surgery may include all or any subset of the following stages, suitably ordered e.g. as shown:
1. Tube placement for L4 TLIF—the tube carries or bears or includes or is marked with a set of fiducial markers that are identified by the system 3D camera aka 3DC and located in space
2. Removal of soft tissue and identification of inferior articulating facet. Typically, following tissue removal, the M3D camera is inserted into or secured to the tube and measures the inferior articulating facet. Using the 3D image of the facet and the location of the tube in space allows locating the full vertebra (in this case L4) in 3D space, and directs the surgeon to adjust the tube if the relative vertebra location requires adjustment.
3. Resection of the inferior facet. During the bone removal process, the M3D camera can be entered into the tube multiple times to measure the bone removal process and provide feedback to the surgeon regarding the amount of bone removed (in 3D) and how close the final bone is to the pre-operational design goal.
4. Resection of superior facet. The superior facet is resected from its tip down to the superior border of the pedicle. Also in this stage, the M3D camera can be entered into the tube multiple times to measure the bone removal process and provide feedback to the surgeon regarding the amount of bone removed (in 3D) and/or how close the final bone is to a pre-operational design goal.

The system software, according to an embodiment of the invention, is now described in detail. The system software may inter alia comprise any software functionalities described in co-owned published PCT patent application PCT/IL2019/050775.

The M3D software may, alternatively or in addition, include all or any subset of the following modules:
1. Depth computation module—acquires depth information from the two cameras 202 and 203. Each camera may be used to get depth information independently e.g. by computing pixel offsets of the pattern projected by projector 204. Pre-calibration of the relative position and/or orientation of the projector and/or each camera, and/or both cameras with respect to each other, can be performed, say, during the M3D camera testing following M3D camera assembly. Each camera 202, 203 image processing software can provide depth of objects in the camera field of view e.g. by converting pixel offsets (computed by cross-correlation of the projected points in the camera image with the original projected point image) of sections of the projected pattern (for example, small groups of semi-random projected dots) to distance from the camera e.g. by triangulation. The computation of depth from pixel offsets may be performed using open source software packages such as OpenCV (https://opencv.org/). The two images from cameras 202 and 203 may be combined to form a continuous 3D depth map of the image area. The software typically combines the depth information from the two cameras, with projector lighting and with non-patterned LED lighting, into a single depth map e.g. high accuracy (<0.3 mm) depth map. The integration of the depth maps allows accuracy that is about X2 better than using only the projected pattern for 3D depth estimation.
2. Integration with global coordinate system—the depth map of the M3D camera is placed in the 3D camera aka 3DC global coordinate system e.g. by taking into account the relative 3D transformation (translation and/or rotation) between the M3D camera and the 3DC camera. The position and/or orientation of the retractor may be measured by the STT connected to the retractor (or other tube), and the M3D camera relative position and/or orientation to the retractor is typically secured or set e.g. by mechanical means that keep the camera locked in a pre-determined position and orientation. The mechanical means may comprise a pin entering a slot, set screw, or any other mechanical setting mechanism known in the art.
3. User interface, registration—the user typically marks the area of bone that is cleared on a pre-operation CT of the patient. For example, in a Laminotomy procedure, this refers to the inferior edge of lamina of the vertebra undergoing surgery, and the extent of tissue removal that exposes the bare bone. The depth map from the M3D camera may be matched to the marked area on the CT and may provide registration between the vertebra CT and the actual position of the vertebra vs. the MIS tube.
4. User interface, bone removal—the user marks a desired amount of bone to be removed on the pre-operation CT. During surgery, each time the user inserts the M3D camera to the tubular retractor, the system typically indicates the amount of bone removed. For example, the software my show the current 3D bone image superimposed on the pre-operational CT image of the vertebra.
5. User interface, retractor motion—the 3D camera aka 3DC continuously tracks the retractor position and orientation (e.g. by tracking the STT) and updates the vertebra position with respect to the retractor accordingly. The user may check the position at any time during surgery by inserting the M3D camera into the retractor tube and performing re-registration of the vertebra.

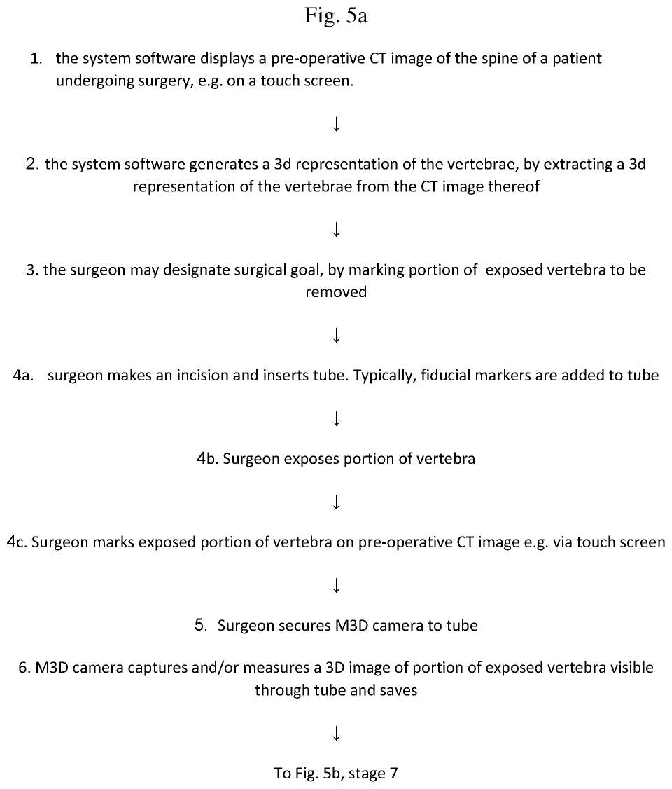

Reference is now made to FIG. 4. FIG. 4 is a schematic drawing of an ultrasound probe with tool tracking unit attached or secured to the probe. The tool tracking unit 200 may be attached to the handle of the ultrasound probe 210. Fiducial markers 205 on the tool tracking unit may be tracked by the 3D camera aka 3DC for accurate positioning of the probe as the probe scans along the patient's spine, allowing registration of the ultrasound image to CT image. An example tool tracking unit is described in the co-owned PCT application whose disclosure is hereby incorporated by reference.

Generally, integration of an ultrasound probe with the system shown and described herein yields enhancement of accuracy and the ability to locate the right position for skin incisions with no additional radiation. The ultrasound scan can be repeated multiple times during surgery to verify registration and navigation.

The surgery flow aka flow 2 with integration of an ultrasound probe e.g. the probe of FIG. 4, may include all or any subset of the following stages, suitably ordered e.g. as follows:
1. Surgeon places sterile fiducial markers (for example, multi-modality markers from https://jrtassociates.com/multi-modalityradiologymarkers.aspx) on patient's skin near expected skin incisions. The markers are used for tracking skin positions by the 3D camera aka 3DC.
2. Surgeon scans patient's back using an ultrasound probe (for example, linear array transducer Ultrasonix L14-5/38 5 MHz probe connected to Ultrasonix SonixMDP scanner) with an attached tool tracking unit. The tool tracking unit includes markers for tracking of the probe position by the 3D camera aka 3DC and feedback to the surgeon to maintain the required angular orientation of the probe—see detailed description and drawing detailing probe attachment to tool tracking unit.
3. Software designed for matching the ultrasound data to pre-op CT data aka US2CT shows successful registration of vertebrae. In case of unsuccessful registration, stages 1 and 2 are repeated.
4. Surgeon makes first incision and inserts the MISS retractor into the incision. The retractor is locked in position, for example by the surgeon activating a mechanical lock mechanism.
5. Surgeon exposes and clears a section of bone including identifiable features through the retractor tube.
6. Surgeon secures the miniature camera onto the tube e.g. inserts M3D camera into retractor tube and locks the M3D camera in position.
7. Surgeon marks the exposed bone feature on pre-operation imagery e.g. CT using system software which may include any software functionality described in the co-owned PCT application.
8. Software performs registration of vertebra— measure bone feature 3D shape from M3D camera combined with 3D position of retractor tube from 3D camera aka 3DC.
9. Software presents to surgeon the retractor superimposed on the pre-operative e.g. CT image with successful registration indication.
10. Surgeon takes out M3D camera from retractor tube to clear tube for other surgery tools (or the M3D design uses such minimal cross section that the camera can remain inside retractor tube during surgery).
11. Surgeon typically shows surgery tools to 3D camera aka 3DC for tool registration and tracking.
12. Surgeon typically verifies vertebra registration by placing tool tip on known bone feature and seeing the software present the tool tip at the known bone feature on monitor showing tool and CT.
13. Surgeon repeats stages 1-6 and 9 for registering each vertebra using all existing retractors.
14. Software and system continuously track the positions and angles of retractors and tools using 3D camera aka 3DC and markers on retractors and tools.
15. Software alerts surgeon to any changes in retractor positions that may affect registration, e.g. detected motion >2 mm (or other chosen threshold).
16. Surgeon repeats stages 3-6 and 9 for any retractor with motion alert to verify proper registration.

Stages 1-3 in flow 2 integrate the ultrasound data with the pre-operational CT for radiation-free vertebrae registration prior to any incision.

The methods of References 4 and 5 herein can be used to perform stage 3 in flow 2. Specifically, the methods and algorithms described in both references can be implemented in the US2CT software in stage 3.

Reference 4 (https://link.springer.com/article/10.1007%2Fs11548-019-02020-1) is a report on matching ultrasound scanning of lumbar spine with CT scanning for posterior bone surface registration. The results show registration errors <2 mm, mostly around 1 to 1.5 mm.

Reference 5 (https://arxiv.org/abs/1609.01483) describes enhancing the accuracy of detection of spinal bone surfaces by using minimum variance beam forming of the ultrasound transducer beam, coupled with phase-symmetry analysis of the images. The authors claim registration to CT image with <1 mm error.

The combination of accurate tracking of the ultrasound probe by the 3D camera aka 3DC, enhanced bone surface contrast using beamforming and optimized detection, with software (aka US2CT, as described in References 4 and 5) that reduces noise and enhances bone structure features—especially posterior cortical bone surfaces of the spinous process and articular processes—will allow sub-millimeter accuracy for registration and navigation during MISS procedures, with no additional radiation to patient or staff.

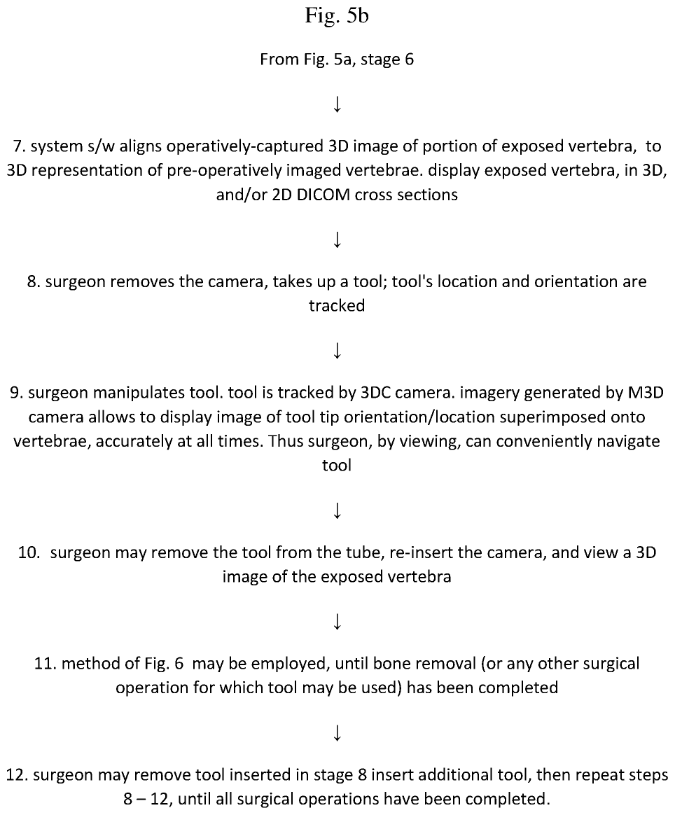

FIGS. 5a-5b, taken together, form a simplified flowchart illustration of a method of operation according to certain embodiments; all or any subset of the following illustrated stages may occur, suitably ordered e.g. as shown.
1. the system software displays a pre-operative CT image of the spine of a patient undergoing surgery, e.g. on a touch screen or other display device which can accept input from a user.
2. the system software generates a 3d representation of the vertebrae, by extracting a 3d representation of the vertebrae from the CT image thereof e.g. as described in the co-owned PCT application.
3. the surgeon may designate a surgical goal, pre-operatively and/or at any point during surgery, by marking, e.g. on the pre-operative CT image or on the 3D representation of the vertebrae generated in stage 2, which portion of the exposed vertebra is to be removed If the goal is pedicle screw insertion, the surgeon navigates the pedicle screw according to a plan e.g. as described in the co-owned PCT patent application and/or using off-the-shelf planning software packages such as, for example http://www.surgimap.com/.
4. surgeon makes an incision and inserts a tube e.g. retractor. Typically, fiducial markers are added to the tube e.g. since each retractor tube typically has a tracker or Surgery Tool Tracking unit aka STT attached thereto, e.g. as described herein with reference to FIG. 3.

The incision is positioned to allow a tool inserted via an outer end, far from the vertebra, of the tube, to perform a surgical operation on (e.g. remove a portion of, clear tissue from, insert a pedicle screw, etc.) the exposed vertebra via an inner (aka bottom) end of the tube, adjacent the vertebra and inside the incision. The tool thus may for example be a bone removal tool and/or a tool to place pedicle screws and/or any other surgical tool.
4b. Surgeon exposes a portion of a vertebra by cleaning tissue therefrom, e.g. via the tube.
4c. Surgeon marks the exposed portion of vertebra on pre-operative CT image e.g. via touching a touch screen presenting the pre-operative CT image of the spine.
   The surgeon may touch just one point on screen, or may trace around the entire area that s/he has exposed, so as to mark the perimeter of the exposed area.
5. Surgeon mounts the M3D camera in or on the tube.
6. The M3D camera captures and/or measures a 3D image of the portion of the exposed vertebra visible through the inner end of the tube (e.g. of the exposed vertebra) and saves this image e.g. as described herein and/or in the co-owned PCT application.
7. The system aligns the operatively-captured 3D image of the portion of the exposed vertebra, to the 3D representation of the pre-operatively CT-imaged vertebrae extracted in stage 2 above, typically including searching a vicinity of the region found in stage 2b until, e.g. via surface matching, a portion of the 3D representation of the vertebrae extracted in stage 2 is found, which portion is similar to a 3D rotation (orientation) and/or 3D translation of the captured 3D image of the body portion. Typically, the system software conventionally computes rigid coordinate transformation (3D rotation and/or 3D translation) between the vertebra and the tube and between the tube and the ‘world’ or global coordinate system of the external 3D camera (3DC); it is appreciated that rotations and translations are both rigid transformations. A display of at least the exposed vertebra, in 3D, and/or 2D DICOM cross sections, may be provided e.g. as described in the co-owned PCT application. The tube's location and orientation may be determined e.g. by virtue of the fiducial markers thereupon.
8. The surgeon removes the camera and takes a surgery tool; the tool's location and orientation are tracked (e.g. by virtue of the fiducial markers)Typically, each surgery tool, whether in the tube or out, bears markers e.g. e.g. as descriebd herein and is tracked by the 3D camera 3DC thus its tip position and orientation is accurately known.

It is appreciated that the fiducial markers may include any feature which is easily detected by image processing. If at least 3 such markers are present in a given image (or, for tracking, in many frames of), the markers' positions allow the tool pose or orientation of the overhead camera or 3DC relative to the features to be derived. Any suitable tool tracking techniques may be employed by the 3D camera (3DC) e.g. as described in the PCT. Typically, when the system tracks an item such as a tool or a tube, the tracked items are connected or secured to or associated with different dynamic reference frames (DRFs) that include fiducial markers tracked by 3DC.

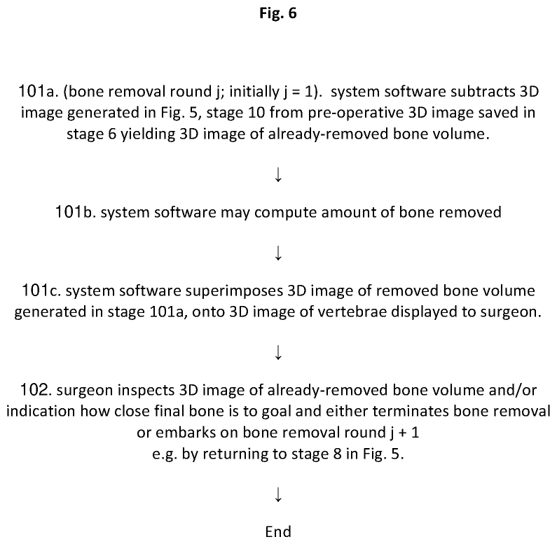

It is appreciated that some (“external”) tools may be tracked even if they are not inserted into the tube, e.g. an awl or screwdriver used to insert a pedicle screw on another area of the vertebrae.
9. The surgeon manipulates the tool. The tool is tracked by the 3DC camera e.g. as described in the co-owned PCT patent application. Advantageously, despite the fact that in an MIS procedure vertebrae are not exposed, the imagery generated by the M3D camera typically allows the system software to continuously superimpose the current tool tip position onto the display generated in stage 7, so as to display an image of the tool tip orientation/location superimposed onto the vertebrae, accurately at all times. Thus the surgeon, by viewing the current position of the tool tip on the screen, can conveniently navigate the tool to where s/he wants to operate and receive real-time feedback on tool tip orientation and location.
10. The surgeon may remove the tool from the tube, re-insert the camera, and view a 3D image of the exposed vertebra (e.g., if the tool is a bone removal tool, of the trimmed vertebra from which some bone has been removed).
11. If the tool inserted in stage 8 is (say) a bone removal tool, and the surgeon's manipulation of the bone removal tool removes some bone from the exposed vertebra, the method of FIG. 6 may be employed, until bone removal (or any other surgical operation for which a given tool may be used) has been completed to the surgeon's satisfaction.
12. The surgeon may remove the tool inserted in stage 8 and insert an additional tool, then repeat stages 8-12, until all surgical operations have been completed to the surgeon's satisfaction.

FIG. 6 is a simplified flowchart illustration of a method of operation according to certain embodiments; all or any subset of the following illustrated stages may occur, suitably ordered e.g. as shown.
101a. (bone removal round j; initially j=1). The surgeon has removed some bone from the exposed vertebra in stage 9; the system software now subtracts the 3D image generated in stage 10 from the pre-operative 3D image saved in stage 6 yielding a 3D image of ‘missing’ or already-removed bone volume.
101b. The system software may compute an amount of bone removed, either a sensed or computed indication of the absolute volume or mass of bone removed, or as a percentage of the target amount of bone to be removed, as defined in stage 3.
101c. The system software superimposes the 3D image of ‘missing’ or already-removed bone volume generated in stage 101a, onto the 3D image of the vertebrae being displayed to the surgeon.
102. The surgeon inspects the 3D image of the already-removed bone volume and/or an indication of how close the final bone is to the pre-operational goal defined in stage 3 and accordingly (e.g. by comparing a current image to a bone removal goal), either terminates bone removal or embarks on bone removal round j+1 e.g. by returning to stage 8 in FIGS. 5a-5b. For example, if a goal including a 3D representation of bone to be removed is defined e.g. as described in stage 3, the system software may subtract the 3D image generated in stage 10 from a pre-operative 3D image on which the 3D representation of bone to be removed, has been superimposed, thereby to provide, for display to the surgeon, a 3D image of not-yet-removed bone volume. Alternatively or in addition, if the total amount of bone to be removed is known (e.g. computation of the volume of a marked region on the pre-op CT) and if stage 101b is performed, the system software may compute a percentage of bone removed thus far.
103. The system software may optionally superimpose the 3D image of ‘missing’ or already-removed bone volume generated in stage 102, e.g. onto the 3D image of the vertebrae being displayed to the surgeon.

It is appreciated that any suitable method may be employed by the system software, to register the original vertebra (e.g. as imaged in the pre-operative CT showing vertebra in pre-operative state, before bone removal or any other surgical operation) to the 3D image which may be generated by the camera in the tube.

Thus the system knows the starting bone structure; a 3D image of the vertebra before bone removal has begun, is available (e.g. may be derived from the pre-operative CT of the vertebra). The new 3D image after bone removal, captured by the camera in the tube each time the surgeon completes all or some of the required bone removal, is registered to a previous 3D image of the vertebra e.g. the most recent available 3D image of the vertebra, just prior to the most recent round of bone removal.

Any suitable method may be used for registration. As described in the co-owned PCT application, typically, the input to registration includes markings, generated by the surgeon who has cleaned tissue off those bone features s/he has chosen as tracking features and marks the “tracking features” on the pre-operative image and on the spine or imagery thereof generated by the miniature camera. In order to establish alignment or registration between the pre-operational scan and the 3D camera images, the bone features extracted from the CT scan and marked on the image may be identified in the 3D camera image. For example, the surgeon may mark a specific point on the patient's spine with a tool typically including or bearing tracking or fiducial markers. The 3D camera tracks the tool tip, and the surgeon may mark the relevant point also on the pre-operative scan data. Even marking only 1 or 2 or 4 or 6 or 10 points per vertebra, is enough to allow system software to match, or align e.g. by surface matching, the pre-operative scan to the 3D camera image. Typically, the surgeon identifies specific points, areas or traces on each vertebra, both on the CT and by marking them to the camera, for example by placing the tip of a suitable tool to each point or by tracing a line on exposed bone area. The software matches the pre-operative image with the 3D positions of the points or traces as seen by the camera, and typically informs the surgeon if there is good registration between the pre-operative imagery and camera image. If the software determines that registration is not high quality, the surgeon may repeat registration, and/or may add additional points, traces or features. For example, the surgeon may expose bone features, clearing away tissue. The surgeon may trace the exposed bone features for the 3D camera using a tracked tool tip, and label the same areas on the pre-operative scan using any suitable computer input device. This way the correspondence or alignment (e.g. registration between the pre-operational scan and at least one 3D camera image generated as surgery begins) between bone features is established, and the 3D image can be registered to the CT scan. The computer or processor or logic may use a suitable registration algorithm including known 3D point cloud registration methods such as Iterative Closest Point (ICP). When the computer signals it has achieved a “good” registration result, for example with Root Mean Square Error (RMSE) fitness better than, say, 0.5 mm between the CT and 3D points, the surgeon can start the operation and the system may use the registration of the bone features for continuous tracking of each individual vertebra.

Once these two images of the same vertebra are registered, the later image may be subtracted from an earlier image, yielding a 3D image of ‘missing’ or already-removed bone volume. Typically, if the vertebra is imaged, say, 5 times, following 5 rounds of bone removal, yielding images 1-5 in addition to the pre-operative image of the vertebra (aka “image 0”), then first, image 1 is aligned or “registered” to image 0, and the system may display the bone removed in round 1 by subtracting image 1 from image 0. The surgeon elects to continue to round 2, typically removes the camera from the tube, and continues bone removal, then returns the camera to the tube and generates image 2. The system aligns image 2 to image 1, but since image 1's alignment to image 0 is known as well, alignment of image 2 to image 0 may be derived, typically using conventional linear algebraic techniques to derive alignment of image 2 to image 0, from the alignment of image 2 to image 1, and from the alignment of image 1 to image 0—. Then, image 2 may be subtracted from image 0 to yield a 3D image of all bone removed thus far (i.e. all bone removed in round 1, plus all bone removed in round 2). This 3D image of missing bone volume (or similar images derived after rounds 3-5 are completed) may be shown to the surgeon superimposed on the pre-operational superimposing may be achieved using any suitable visualization scheme such as, for example, rendering one of the images semi-transparent and/or adding color to the difference.

The surgeon may observe the images (images 1-5 in the above example) visually presenting the surgical progress e.g. the extent of removed bone, and decide when to stop (e.g. when to stop removing bone). In the above example, if the surgeon is satisfied with image 5, the surgeon will not embark on a bone removal round 6, and will instead close the surgical field and terminate surgery.

### EXAMPLES

Tubular Laminotomy may be performed, wherein soft tissue is removed and, subsequently, an inferior edge of at least one lamina and/or ipsilateral base of spinous process having been identified, the M3D camera is secured to a top end of the tube, and the inferior edge of the lamina, as viewed through the bottom end of the tube, is measured. Then, the full vertebra's 3D location is derived e.g. by matching of the measured surface to the marked surface of this area from a 3D image of the lamina edge and from the tube's known (due to the fiducial markers on the tube) location in space.

According to another example, MIS TLIF (Transforaminal Interbody Fusion) is performed, including removal of soft tissue and identification of the inferior articulating facet, followed by securing the M3D camera to a top end of the tube and measurement by the 3D camera of the inferior articulating facet, as viewed through the bottom end of the tube. Then, the full vertebra's 3D location is derived from a 3D image of the facet and from the tube's known (due to the fiducial markers on the tube) location in space.

The system may direct the surgeon to adjust the tube if the vertebra location, relative to axes marked by the surgeon, requires adjustment. Typically, the system software generates a display of a tool tip which helps the surgeon determine if and how to move the tool.

A particular advantage of certain embodiments is that an adequate depth of field for mini-surgery use cases is achievable e.g. if it is desired to achieve a depth of plus/minus 5 mm (total 10 mm) which is achievable by a surgeon, and/or corresponds to or takes into account, a maximum vertical distance between various features of a portion of human bone whose diameter is the diameter of a tube e.g. up to 25 mm. This may be achieved, in the embodiments herein, by providing a suitable tube length, one which provides a distance an order of magnitude which is larger (×10) e.g. 50 mm between the near end of the tube, where the camera is mounted, and the far end of the tube.

References to CT herein are merely by way of example. Instead, any scan modality which yields 3D information regarding bone surface of vertebrae may be employed, such as but not limited to MRI, ultrasound, combination of 2D X-ray images (fluoro), combinations of any of the above, or other known alternatives.

According to certain embodiments, the system software is operative to recognize a location of the scene within a larger topology e.g. a 3D representation of one or more vertebrae. Typically, mapping a 3D topology imaged by the M3D camera to bone surface extracted from pre-operative imagery, e.g. CT scan/s, provides relative coordinates of vertebra with respect to the M3D camera. Conversion to ‘world’ coordinates may be performed by using the known location and orientation of the M3D camera provided by the 3DC system camera that tracks the tubular retractor and the surgery tools. Typically, the surgeon marks the exposed bone surface and this marked area is used for registration or alignment e.g. as described herein. It is appreciated that extraction of bone features from pre-operational imagery or conversion of pre-operative imagery into 3D structures may be performed in accordance with any of the teachings in the co-owned PCT application. Surface matching may be used to best match a surface measured by a 3D camera during surgery, to a typically larger and three-dimensional bone surface extracted from a pre-operative image, such as but not limited to a CT image.

Any suitable method may be employed for conversion of pre-operative imagery into 3D structures. The areas used for registration or alignment (e.g. by surface matching) may include whatever is visible (e.g. to the miniature camera) via the tube. The surgeon typically marks the exposed area on the pre-operation imagery and this is the basis for registration or alignment which may, as described in the published PCT application, include all or any subset of: i) vertebrae bone features for real time tracking; ii) extraction of bone features from pre-operational imagery; and iii) registration & tracking of individual vertebrae. The individual features to be tracked vary between surgeries. For example, the area for registration may be a lamina's inferior edge, or may be an inferior articulating facet.

It is appreciated that terminology such as “mandatory”, “required”, “need” and “must” refer to implementation choices made within the context of a particular implementation or application described herewithin for clarity and are not intended to be limiting, since, in an alternative implementation, the same elements might be defined as not mandatory and not required, or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations or stages of any of the methods shown and described herein, in any suitable order, including simultaneous performance of suitable groups of operations as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g. in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g. by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network, e.g. web-based system employing software, computers, routers and telecommunications equipment as appropriate.

Any suitable deployment may be employed to provide functionalities e.g. software functionalities shown and described herein. For example, a server may store certain applications, for download to clients, which are executed at the client side, the server side serving only as a storehouse. Any or all functionalities e.g. software functionalities shown and described herein may be deployed in a cloud environment. Clients, e.g. mobile communication devices such as smartphones, may be operatively associated with, but external to, the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false and to perform y each time x is determined to be true, thereby to yield a processor which performs y at least once, typically on an “if and only if” basis e.g. triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may, for example, comprise changing the state or condition, or may more generally cause any outcome which is technically advantageous given the state or condition or data, and/or may prevent at least one outcome which is disadvantageous given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art and particularly, although not limited to, those described in the Background section or in publications mentioned therein.

Conversely, features of the invention, including operations, which are described for brevity in the context of a single embodiment or in a certain order, may be provided separately or in any suitable sub-combination, including with features known in the art (particularly, although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein e.g. wired data communication and/or in short-range radio communication with sensors such as cameras e.g. via WiFi, Bluetooth or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable or facilitate that operation or to enable, facilitate or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry, including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties , logic, modules, blocks, operations or functionalities described herein, which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element e.g. operation described herein, may have all characteristics and attributes described or illustrated herein, or, according to other embodiments, may have any subset of the characteristics or attributes described herein.

1.-30. (canceled)

31. An imaging system aka 3d camera operative in conjunction with a tube (e.g. retractor or trocar) having two open ends, the system comprising:
active portions small enough to fit into the tube; and
an electronic subsystem including a hardware processor operative to receive at least one image from said active portions and to generate therefrom at least one 3D image of a scene (aka miniature scene) visible via one of the tube's open ends (aka portion of a surgical field aka topology).

32. The system of claim 31 and also comprising a tracker configured to be secured to the tube, thereby to monitor an absolute location of the retractor.

33. The system of claim 31 and wherein said active portions comprise:
at least one image sensor/s or cameras oriented to have a partially or totally overlapping field of view, and
at least one structured light projector/s projecting a known pattern onto the field of view of the image sensor/s.

34. The system of claim 31 and wherein at least one dimension of said active portions is smaller than the tube's inner diameter.

35. The system of claim 31 which includes at least one component which is larger in size than the tube's inner diameter.

36. The system of claim 31 and wherein the imaging system includes at least one mechanical subsystem configured to secure the camera at a fixed location and orientation vs. markers that track the tube.

37. The system of claim 33 and wherein the hardware processor receives data from the at least one image sensor and generates said 3D image from said data.

38. The system of claim 37 and wherein at least one image sensor is deployed at an offset from at least one structured light projector
and wherein the offset is known to the hardware processor and is used for triangulation which generates said 3D image from said data.

39. The system of claim 31 and wherein the hardware processor assigns absolute coordinates to the 3d image of the surgical field.

40. The system of claim 31 and wherein the hardware processor is also configured to monitor a tool which is tracked, hence its absolute coordinates are known, and is moving.

41. The system of claim 31 and wherein the hardware processor is configured to recognize a location of the scene within a larger topology e.g. a 3D representation of one or more vertebrae.

42. The system of claim 31 and wherein said tool is deployed inside the retractor.

43. The system of claim 31 and wherein said tool is deployed outside the retractor.

44. The system of claim 31 and also comprising at least one tracker attached to the tube, and wherein the hardware processor is configured to use data from the tracker to be presented to a human user, thereby enabling the human user to monitor a current position of the tube.

45. The system of claim 31 and wherein the hardware processor is configured to superimpose the 3D image of the miniature scene onto an earlier captured image of a larger scene which is larger than, and includes, the miniature scene, thereby to generate a superimposed image, and to display the superimposed image to a human user.

46. The system of claim 36 wherein the mechanical subsystem is larger in size than the tube's inner diameter.

47. A computer program product, comprising a non-transitory tangible computer readable medium having computer readable program code embodied therein, said computer readable program code adapted to be executed to implement An imaging method operative in conjunction with a tube having two open ends, the method comprising:
receiving at least one image from active portions, of a 3d camera, which are small enough to fit into the tube and
using a hardware processor to generate therefrom at least one 3D image of a scene.

48. The system of claim 38 wherein said at least one image sensors comprises two image sensors, and wherein said triangulation comprises stereo triangulation.

49. The system of claim 38 wherein said at least one projector comprises but a single projector, said at least one image sensor comprises but a single image sensor, and wherein pattern correlation and measurement of sub-pattern displacement are used for said triangulation or for depth estimation.

50. The system of claim 41 and wherein at least one pre-operative image, having a resolution, represents the larger pre-mapped topology, and wherein the pre-operative image comprises a CT image.

51. An imaging method operative in conjunction with a tube having two open ends, the method comprising:
providing a 3d camera with active portions small enough to fit into the tube and
using an electronic subsystem including a hardware processor operative to receive at least one image from said active portions and to generate therefrom at least one 3D image of a scene.

52. The method of claim 51 and wherein the tube bears fiducial markers and wherein the tube's location in space is known to said hardware processor due to said markers.

53. The method of claim 52 and wherein at least when an inferior edge of at least one lamina and/or ipsilateral base of spinous process are identified, the 3d camera is secured to a top end of the tube, and the inferior edge of the lamina, as viewed through the bottom end of the tube, is measured, thereby to yield a measured surface; and wherein at least one vertebra's 3D location is presented to a human user, thereby to facilitate performance of Tubular Laminotomy.

54. The method of claim 53 wherein said vertebra's 3D location is derived by matching the measured surface to a portion of a 3D image of at least a portion of the lamina and from the tube's known location in space.

55. The method of claim 52 wherein the camera is secured to a top end of the tube and measures an inferior articulating facet, as viewed through the bottom end of the tube, and wherein at least one vertebra's 3D location is presented to a human user, thereby to facilitate performance of MIS TLIF (Transforaminal Interbody Fusion).

56. The method of claim 55, said vertebra's 3D location being derived from a 3D image of the facet and from the tube's known location in space.

57. The system of claim 31 and also comprising at least one tool tracker and wherein the hardware processor is configured to use data from the tracker to be presented to a human user, thereby enabling the human user to monitor a current position of the tool.
